(19) United States
(12) Patent Application Publication
(10) Pub. No.: US 2010/0269578 A1
(43) Pub. Date: Oct. 28, 2010

(54) METHOD TO CHARACTERISE ROCK FORMATIONS AND APPARATUS FOR USE THEREWITH

(75) Inventors: Emmanuel Detournay, Roseville, MN (US); Andrew Bunger, Victoria (AU)

Correspondence Address: NIXON & VANDERHYE, PC, 901 NORTH GLEBE ROAD, 11TH FLOOR, ARLINGTON, VA, 22203, US

(73) Assignee: COMMONWEALTH SCIENTIFIC AND INDUSTRIAL RESEARCH ORGANISATION, CAMPBELL (AU)

(21) Appl. No.: 12/734,776

(22) PCT Filed: Nov. 24, 2008
(86) PCT No.: PCT/AU2008/001738

§ 371 Date: Jun. 23, 2010

(30) Foreign Application Priority Data
Nov. 23, 2007 (AU) 2007906430

(51) Int. Cl. G01N 33/24

(52) U.S. Cl. 73/152.11

(57) ABSTRACT

A portable apparatus (111) to measure the change in behaviour of a rock specimen (113) in a fluid (25) of prescribed composition. The rock specimen (113) is prepared from drill cuttings, wellbore cave-in material, or core offcuts returned during the drilling operation of a wellbore. The apparatus (111) comprises a vessel (23) in which the fluid (25) and specimen (113) are placed. The apparatus (111) has a loading means for applying at least one load upon the specimen (113) and at least one measuring device (119) to measure the deformation of the specimen (113) in response to a change in load. The apparatus (111) also comprises a stirring system to maintain uniformity of the fluid (25) and a temperature regulating device (131) to maintain the fluid (25) at the desired temperature.

### FIELD OF THE INVENTION

The present invention generally relates to rock properties. In particular this invention relates to predicting the mechanical behaviour of a rock formation when exposed to an aqueous fluid.

### BACKGROUND ART

Drilling wells for the extraction of oil and/or gas from a reservoir is one of the most basic necessities of the petroleum industry. During the drilling process a sophisticated fluid, known as a drilling mud is circulated in the wellbore in order to lubricate the drilling bit, carry debris out of the wellbore, and provide temporary support to the wellbore itself due to the hydrostatic pressure created by the fluid.

Shale formations comprise perhaps the most commonly encountered rock type in petroleum industry drilling operations. Shale is generally characterized as being fine grained, with a high clay content, has fine pores and a low-permeability matrix. Because shales have high clay content, they interact strongly with water-based fluids. As a result drilling through shale is often slow and difficult.

When drilling through shale a variety of problems are common including wellbore closure, collapse and erosion. Furthermore, if the quality of the bore is compromised then further difficulties will also be experienced when positioning the casings therein.

The petroleum industry has long relied on oil-based drilling fluids, particularly when drilling through shale formations. However, the environmental constraints and associated costs which surround the disposal of oil-based waste products are driving a trend towards the use of water-based drilling fluids.

A major technological concern with water-based drilling fluids arises from the interaction of the fluids with shales which, along with mudstones, siltstones, and claystones, comprise 75% of drilled sections in oil and gas wells and cause 90% of the drilling problems related to wellbore instability.

Changes in the stress state of the well are influenced by the physicochemical interaction between the drilling fluid and the formation pore fluid (i.e. exchange of water molecules and solute ions). The presence of water in the drilling fluid induces swelling in the shale, hence changing the stress state of the rock surrounding the wellbore and often reducing the diameter of the wellbore through local failure of the rock.

A means for improving wellbore stability is to add various salts to the drilling fluid. This has the effect of drawing water out of the rock and stabilising the formation around the well. Success of this approach relies on the extent to which the (coupled) pore pressure and stress fields in the immediate vicinity of the wellbore are caused to alter.

The extent to which wellbore remains stable depends on the shale possessing certain chemo-mechanical properties and will vary according to the degree to which these properties are inherent in the shale.

The current state of research involves viewing shales as partial ion exclusion membranes and then characterizing shales via a parameter referred to as membrane efficiency or reflection coefficient. Existing methods for determining the reflection coefficient which are based on pore pressure transmission tests are often uneconomical. This is due to the need to delay normal drilling operation in order to extract a core, combined with the long duration per experiment (usually a few days) and high failure rate of the usual pore pressure transmission tests.

The preceding discussion of the background to the invention is intended only to facilitate an understanding of the present invention. It should be appreciated that the discussion is not an acknowledgment or admission that any of the material referred to was part of the common general knowledge at the priority date of the application.

### SUMMARY OF INVENTION

It is desirable to be able to predict the stability of the rock formation surrounding a wellbore, and its behaviour with changes in mechanical, hydraulic and/or chemical loading applied thereto.

It is an object of this invention to provide a method and apparatus to measure the properties of a rock formation (e.g. shale) such that the behavior of a well may be better predicted.

It is a further object of this invention to provide a method to ascertain a detailed chemoporomechanical characterisation of shale formations in a manner which is economical, non-disruptive to drilling operations, and which is sufficiently rapid.

Once the shale is characterized the results can be used as the basis for decisions regarding drilling fluid composition. This is one of the most important considerations for maintaining wellbore stability during the drilling process.

It is a further object of the invention to provide a portable, autonomous apparatus to enable experiments using a cutting from the drilling operations (cuttings can be collected from the drilling fluid which is returned to the surface). Combined with an integrated method for parameter identification based on experimental data, this device will provide vital input which will allow an accurate engineering decision to be made within 24 hours from the first encounter with a particular formation.

The present invention provides a method for characterising a rock formation. Within this method an apparatus is used and a method applied to measure the change in behaviour of a rock sample when subjected to certain changes in mechanical, hydraulic, and/or chemical loading.

The resulting data relating to the behavioural changes may then be used in a model and a solution developed for that sample. A solution for the appropriate sample geometry and loading applied thereto may then be fitted to the experimental data. The chemoporoelastic parameters including the drained elastic compliance (C), Poisson's ratio (ν), hydraulic diffusivity (Dₕ), ionic diffusivity (Dₛ), chemomechanical coupling parameter (α), and reflection coefficient (R), that make the solution most closely match the data can then be taken to characterise the rock. Applying these results to a simple wellbore stability analysis provides a relative quick and simple means in which drilling engineers can identify what the density and/or salt content of the drilling fluid is required in order to improve wellbore stability. As this can be done relatively quickly with the current invention, a drilling engineer may easily repeat this method and adjust the drilling fluid accordingly when each problematic shale formation is encountered during drilling operations. Furthermore, the chemoporoelastic parameters of the rock that are identified by this invention are essential for predicting wellbore stability.

The present invention provides a method of predicting the behaviour of a rock formation when exposed to changes in chemical composition and/or pressure of a drilling fluid, the method comprising:
using a portable apparatus measure the displacement of a specimen of the rock formation in a fluid over a period of time in which a load is applied thereto, that load being in the form of a mechanical force applied to the specimen and/or the addition of salt to the fluid;
using these measurements and known formula to determine various chemoporomechanical properties of the rock formation;
using formulas that predict wellbore stability and depend on the determined chemoporomechanical properties of the rock formation in order to recommend optimal modifications to the salt content and/or density of the drilling fluid in order to promote wellbore stability.

### Method for Measuring the Behaviour of a Rock Sample

The present invention provides a method for measuring the change in behaviour of a rock specimen in a fluid of prescribed composition, wherein the rock specimen is prepared from drill cuttings, wellbore cave-in material, or core offcuts the method comprises:
stirring the fluid so that the salinity level of fluid remains substantially uniform;
controlling the temperature at which the experiment is conducted such that it is maintained within the desired range;
applying at least one load to the specimen;
measuring the deformation of the specimen in response to the change in the at least one load.

Preferably the method comprises an initial step of preparing a specimen from drill cuttings or other small shale fragments. The specimen may be a regular geometrical shape, such as a cylinder, sphere, disc, or may be of irregular geometry.

Preferably the means employed to stir the fluid is such that effects of the electromagnetic field and other forms of interference upon the measuring instruments is minimised.

Preferably the method comprises an initial step of allowing sufficient time for the specimen to equilibrate with the fluid. Preferably this period is sufficient time for the specimen to reach equilibrium, evidenced by the length of the specimen remaining substantially constant. This period may be in the order of 10-60 minutes. In most cases the fluid's initial composition will have a slightly different composition to the pore fluid of the specimen. As a result there will be a degree of ionic and fluid transfer when the specimen is first placed in the fluid. Once this dissipates the specimen and fluid are in an equilibrate state. Also there may be some thermal equilibration that happens during this initial stage.

Preferably the changes in loading upon the specimen may be in the form of applying a chemical and/or mechanical load thereto. The changes in loading may be changes in the chemical composition of the fluid, preferably by adding salt, and/or changes in the mechanical loading applied to the specimen. Preferably chemical loading is accompanied by the application of a mechanical load.

In one aspect of the invention the method comprises the step of adding salt (e.g. NaCl) over a period of time to alter the chemical loading by changing the ionic concentration. This period may be in the order of 30-60 seconds. Initially a change in salt content in the fluid results in a rapid contraction of the sample over approximately a 30 minute period. Over the next hour this contraction partially recovers. Further salt may be added and measurements continue to be taken over the next 5 to 15 hours. At the end of this period the complete data set has been recorded. A solution can then be developed for that sample using the model.

In those aspects of the invention in which mechanical loading is required a force is applied directly to the specimen. The force may be applied by a weight applied to the specimen. The mechanical loading may begin with a series of axial loading wherein the load is removed and replaced in quick succession. The purpose of this initial series is to ensure complete contact between the specimen and loading surface is established. The mechanical loading may then comprise applying a load, measuring the initial elastic response and measuring the change in displacement over the next one to three hours. During this period water is expelled from the specimen as a result of the applied load. The displacement measured during this period provides information about the elasticity parameters and permeability of the sample.

The change in load to the specimen may also be in the form of applying a hydraulic load to the specimen.

In those aspects of the invention in which hydraulic loading is required, hydraulic pressure is applied upon placing the specimen, fluid and measurement apparatus in a suitable pressure vessel.

Preferably the temperature is maintained a few degrees above ambient or at the in situ temperature of the shale formation.

### Apparatus

The present invention provides a portable apparatus to measure the change in behaviour of a rock specimen in a fluid of prescribed composition, wherein the rock specimen is prepared from drill cuttings, wellbore cave-in material, or core offcuts, the apparatus comprises:
a vessel in which the fluid and specimen are placed;
a loading means for applying at least one load upon the specimen;
at least one measuring device to measure the deformation of the specimen in response to a change in load;
a stirring system to maintain uniformity of the fluid;
a temperature regulating device to maintain the fluid at the desired temperature.

Preferably the apparatus also comprises a data acquisition system to record the data relating to the deformation of the specimen.

Preferably the apparatus is self contained and autonomous.

Preferably the measuring device and data acquisition system is shielded to prevent noise from other devices affecting the data.

Preferably the stirring system is sufficiently shielded to minimise the electromagnetic field and other forms of interference upon the measuring device and data acquisition system.

Preferably the fluid used in the vessel initially approximates the composition of the specimens natural pore fluid.

The specimen may be a regular geometrical shape, such as a cylinder, sphere, disc, or may be of irregular geometry. Preferably the specimen has one or more axis of symmetry. A specimen of a regular geometric shape allows the method of characterising the rock formation to use tractable formulas. By using an irregular shape one simplifies specimen preparation and potentially makes the test sensitive to a wider range of chemoporoelastic parameters, but computationally expensive and time consuming numerical methods must be used. Hence, the use of simple specimen geometries are favoured as this simplifies and speeds up the interpretation of the experimental data.

Preferably the specimen is cylindrical. The specimen may have a diameter of approximately 4 mm. The specimen may be prepared in a jig from a drill cutting.

The loading means may apply a chemical and/or mechanical load to the specimen. The load may be maintained at the required level.

The chemical load may be applied by changing the ionic concentration of the fluid. This may be done by adding salt to the fluid.

The mechanical load may be applied by applying a force to the specimen. This may be done by applying a weight to the specimen. The weight may be applied axially with respect to the specimen. By doing this the deformation of the specimen is constrained so that the specimen remains in substantially the same shape as it shrinks or swells during the test, thus keeping the specimens shape consistent with that which is analysed.

The loading means may also apply a hydraulic load to the specimen. The hydraulic load may be applied by applying a pressure to the fluid in which the specimen is located. Preferably the loading means to change the hydraulic loading comprises a pressure chamber, wherein the vessel, fluid, specimen and measuring device are placed within the pressure chamber.

Preferably the at least one measuring device is capable of measuring a micrometer-scale response on a specimen less than 5 mm in thickness

The at least one measuring device may comprise a linear variable differential transformer. Preferably the linear variable differential transformer comprises a loading platen through which the mechanical loading may be applied to the specimen. The loading platen may be connected to a loading arm to which the desired weight may be applied. By having the loading platen incorporated within the linear variable differential transformer, the repeatability and quality of the contact with the specimen is improved. Obviously other measuring devices may be used to measure the deformation of the specimen.

In one aspect of the invention the temperature regulating device comprises heat elements located in the wall of the vessel.

In another aspect of the invention the temperature regulating device comprises heat elements located in the fluid.

The present invention further provides a portable measurement apparatus which can be used in the laboratory or transported to drilling sites in order to perform on-site characterisation of shale formations.

Preferably the apparatus includes a means for applying a prescribed ionic, mechanical and/or hydraulic loading to specimens, with stirring and temperature regulation as appropriate.

The measurements made by said apparatus can be interpreted in order to provide detailed characterisation of the chemoporoelastic parameters associated with the shale formation. These parameters are then suitable as input for wellbore stability models to assist in engineering decision making.

The present invention provides a method to identify wellbore stability wherein a reflection coefficient and chemomechanical coupling parameter (α) of a rock formation is determined. Preferably these parameters, once determined, can be used to predict the effects of adding salt to the drilling fluid. Preferably the reflection coefficient also allows an operator to determine the desired salt concentration of the drilling fluid.

Preferably the method also determines a chemomechanical coupling parameter α allowing an operator to determine the long term stability of the rock formation (e.g. shale). When the chemomechanical coupling parameter α is a small value the rock formation would be expected to recover nearly all of its original shrinkage.

### The Calculations

The measurement method is predicated on a thermodynamically consistent, fully coupled theoretical framework provided by linear chemoporoelasticity. Herein the response of a shale body to a change in the mechanical loading, the surrounding hydraulic pressure, or the ionic content of the surrounding fluid can be determined provided one has the appropriate values for seven parameters, which appear in the chemoporoelastic constitutive equations and three parameters which appear in a generalized form of Fick's/Darcy's law.

Specifically, we begin with the constitutive equations relating the volumetric strain ε, Biot's fluid content ζ, and salt content θ to the mean Cauchy stress σ, the hydraulic pressure pₕ, and the osmotic pressure p_{c}. Note that the osmotic pressure is given according to the van't Hoff law by p_{c}=R_{g}Tcₛ, where Cₛ is the molar salt concentration, R_{g} is the gas constant (8.13 J/mol Kelvin), and T is the absolute temperature. The volumetric response can hence be expressed as

$$( \begin{matrix}ɛ \\ ζ \\ θ\end{matrix} ) = C ( \begin{matrix}σ \\ {p}_{h} \\ {p}_{c}\end{matrix} ) , C = ( \begin{matrix}C & bC & - α bC \\ bC & {S}_{σ} & - {S}_{σ} β \\ - α bC & - {S}_{σ} β & γ\end{matrix} ) . \qquad ( 1 )$$

The compliance matrix C is comprised of six parameters with a symmetry that is required by Maxwell's equations for the existence of an energy potential. The drained volumetric compliance is given by C (for shales C≈10⁻³ 1/MPa).

The classical poroelastic parameters b and S_{σ} are the Biot stress coefficient (for shales b≈1) and the unconstrained specific storage coefficient S_{σ}=bC/B where B is Skempton's pore pressure coefficient (for shales B≈1).

The parameters α,β∈[0,1] quantify the chemomechanical and chemohydraulic coupling, respectively. Note that α=β=0 corresponds to the absence of chemical coupling and α=β=1 corresponds to immobility of the salt ions.

Finally, γ is a chemical parameter that is given for a saturated porous medium by:

$$γ = \frac{φ}{{R}_{g} T {\overline{c}}_{s} ( 1 - {\overline{c}}_{s} {v}_{s} )} ,$$

where φ is the rock porosity, νₛ is the molar volume of the salt (νₛ=0.03 Liter/Mol for NaCl), and c̅ₛ is a mean salt concentration.

The above volumetric response (1) implies extensions of the tensorial elasticity equations, which can be expressed in terms of the cartesian coordinates {x₁, x₂, x₃} as

$$\begin{matrix}2 G {ɛ}_{ij} = {σ}_{ij} - \frac{3 v}{1 + v} {σδ}_{ij} + \frac{1 - 2 v}{1 + v} b {δ}_{ij} ( {p}_{h} - α {p}_{c} ) & ( 2 ) \\ {σ}_{ij} = 2 G {ɛ}_{ij} + \frac{2 Gv}{1 - 2 v} {ɛδ}_{ij} - b {δ}_{ij} ( {p}_{h} - α {p}_{c} ) . & ( 3 )\end{matrix}$$

Here G is the shear modulus, ν is Poisson's ratio, δᵢⱼ is the Kronecker delta, and henceforth summation on 1 to 3 is implied by repeated indices unless otherwise noted. One may also define σ=σₖₖ/3, ε=εₖₖ, with summation from 1 to 3 on the repeated index, and we recall that the elastic moduli relate as C=3(1−2ν)/2G(1+ν).

The constitutive description of the shale material also includes equations related to the transport of fluid and ions in the porous shale material. These transport processes are accounted by introduction of the so-called reflection coefficient R∈[0,1] to obtain generalized Darcy's and Fick's laws.

$$( \begin{matrix}q \\ r\end{matrix} ) = {SD}_{h} R ( \begin{matrix}∇ {p}_{h} \\ ∇ {p}_{c}\end{matrix} ) , R = ( \begin{matrix}1 & - R \\ - R & \frac{{D}_{s}}{{D}_{h} SN}\end{matrix} ) , \qquad ( 4 )$$

Here q is the specific discharge of the fluid relative to the solid and r is the specific discharge of salt relative to the solvent.

Additionally the hydraulic diffusivity can be expressed as Dₕ=k/Sμ, with k the intrinsic permeability, μ the dynamic fluid viscosity, and S the classical specific storage given by:

$$S = - {b}^{2} C + {S}_{σ} + \frac{b η}{G} . \qquad ( 5 )$$

Finally, Dₛ is an apparent chemical diffusion coefficient and N=R_{g}T/νₛ is a characteristic chemical stress.

The mathematical formulation then requires conservation laws governing linear momentum and fluid and ion mass balance. Linear momentum balance can be stated as:

σ_{ij,j}=−Fᵢ, (6)

where Fᵢ is the i^{th} component of the body force and f_{,j} indicates differentiation of f with respect to the j^{th} coordinate.

Additionally, for an incompressible fluid with no fluid or ion sources inside the body, the mass balance equations are

∇·q=−\dot{ζ}, ∇·r=−\dot{θ}, (7)

where the over-dot indicates absolute differentiation with respect to time t.

Finally, the governing equations require the usual relations between the strain tensor ε and the displacement u from linear elasticity, that is:

$${ɛ}_{ij} = \frac{1}{2} ( {u}_{i , j} + {u}_{j , i} ) , \qquad ( 8 )$$

where uᵢ is the i^{th} component of the displacement.

In all problems the shale body is considered to be at equilibrium at time t=0 so that {σ, pₕ, p_{c}, ε, ζ, θ} may all be understood as perturbations to this initial state which can arbitrarily be taken as zero, i.e. the initial conditions are:

σ=pₕ=p_{c}=ε=ζ=θ=0, t=0. (9)

Hence, the problem formulation is completed by the addition of boundary conditions appropriate to a given problem. In general, solutions require numerical methods, however semi-analytical solutions have been derived for some idealized geometries.

The general principle behind experimentation is then to devise a method by which one or more chemoporomechanical quantity can be measured at one or more locations on or within a specimen which is subjected to some form of loading. Examples could include measurement of the mechanical stress developed at a fixed boundary or measurement of the displacement at some location of the specimen. Some subset of the parameters
{Dₕ, Dₛ, R, C, B, b, α, β, γ, ν}
   is then estimated based on fitting a solution for an appropriate geometry and loading to the experimental data. Here we specifically propose an experimental method in which the displacement of the surface of small specimens, for example which have been prepared from cuttings of <5 mm that are a byproduct of drilling operations, is measured at one or more locations as the specimen is subjected to a change in hydraulic pressure pₕ, osmotic p_{c}, and/or a mechanical loading applied at its boundary.

An appropriate solution to the chemoporoelastic model is then deployed in an inverse problem in order to select material parameter values which produce the best fit between the model and the data.

The present invention further provides a method by which one or more chemoporomechanical quantity(ies) can be measured at one or more locations on or within a specimen which is subjected to a form of loading. Preferably the quantity(ies) measured include the mechanical stress developed in the specimen at a fixed boundary, or a measured displacement field.

### Method for Predicting the Stability of a Wellbore

The present invention further provides a method to predict the stability of the rock formation surrounding a wellbore, the method comprises the steps of:
expressing the experimental data in terms of appropriate quantities in an experimental configuration;
solving a chemoporoelastic boundary value problem relevant to the experimental configuration;
employing said solution in an inverse problem to select material parameters which give the best agreement between the experimental configuration results and the experimental data;
evaluating the uncertainty associated with said parameter estimates and possible covariance between experimental configuration parameters for a given experiment so that the possible error introduced into the wellbore stability calculation by this uncertainty can be quantified; and
using a wellbore stability analysis to provide recommendations regarding the anticipated response of the rock formation to the addition of salt to the drilling fluid.

The present invention has been devised so that a rock formation may be characterised within a 24 hour period from a drill bits first encounter with a new formation. In order to achieve this outcome it is necessary to provide means in which a sample of the drilling cuttings (or similar) can be analysed and characterised quickly. This is achieved by providing a test apparatus which can be used at site, is portable and autonomous but which can also measure changes in a small specimen whilst load conditions acting thereupon are applied. Once the rock has been characterised, the resulting chemoporoelastic parameters may then be used in a wellbore stability model to determine what changes are required to salt concentration of the drilling fluid and/or the pressure of the drilling fluid to achieve wellbore stability.

### BRIEF DESCRIPTION OF THE DRAWINGS

The invention will be better understood by reference to the following description of several embodiments thereof as shown in the accompanying drawings in which:

FIG. 1 is a schematic of an apparatus according to a first embodiment of the invention;

FIG. 2 is a graph representing experimental results shown with model results using fitted parameters according to an example which utilises an embodiment of the invention;

FIG. 3 is a schematic of an apparatus according to a second embodiment of the invention;

FIG. 4 is a modelled view of the apparatus shown in FIG. 3;

FIG. 5 is a modelled view of a series of apparatus as shown in FIG. 4, in a transportable case;

FIG. 6 is a graphical representation of change in a specimen length for three stages of loading according to a second embodiment of the invention; and

FIG. 7 is representation of a wellbore stability model at the wall of a wellbore.

### BEST MODE(S) FOR CARRYING OUT THE INVENTION

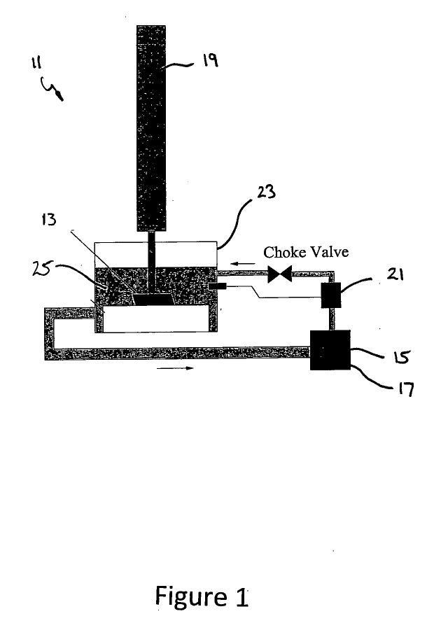

FIG. 1 illustrates the basic components of an apparatus 11 to measure the deformation of a rock specimen 13, according to a first embodiment of the invention.

The apparatus 11 incorporates a stirring system 15 which comprises a circulation pump 17. The circulation pump 17 is designed so as to be placed well away from other electronic components so that measurements of a small amplitude response (e.g. in the order of a few micrometers) are not affected by noise caused by the stirring system 15.

Also in this embodiment there is a measuring device which is in the form of a single, hermetically-sealed direct current displacement transducer (DCDT) 19, driven by a high precision regulated power supply. This measures the change in thickness of a disc, cylinder, or irregularly shaped specimen 13 located in a vessel 23.

The specimen 13 is located in a fluid 25 within the vessel 23. The fluid 25 approximates the composition of the specimens 13 natural pore fluid.

The temperature of the fluid 25 is regulated by a feedback loop controlled in-line heater 21 which, in other embodiments, could be replaced by a constant temperature cabinet or heating elements that surround the vessel 23.

After allowing some time (approximately 10-60 minutes) for the specimen to equilibrate with the fluid 25, changes in ionic concentration are incurred by a steady addition of salt (e.g. NaCl) over a short period of time, typically lasting from 30 to 60 seconds.

Hydraulic pressure changes are applied upon placing the vessel and measurement apparatus in a suitable pressure vessel. The pressure vessel is then pressurized with air up to a desired level (approximately 10-20 MPa) so as to raise the fluid pressure without subjecting the transducers to corrosive ionic solutions.

Experimental duration is governed by the specimen size and the hydraulic and chemical diffusivities (Dh and Ds) and is typically 4 to 20 hours.

The experimentation and analysis thus entails:
1. Measurement of the displacement of the specimen boundary due to the shrinking/swelling induced by a change in the composition or pressure of the surrounding fluid.
2. Solving a chemoporoelastic boundary value problem relevant to the experimental configuration to predict the displacement of the boundary that was measured in the experiment.
3. Employing said solution in an inverse problem to select material parameters which give the best agreement between the model results and the experimental data.
4. Evaluation of the uncertainty associated with said parameter estimates and possible covariance between model parameters for a given experiment.
5. Providing recommendations regarding the anticipated response of a shale to the addition of salt to the drilling fluid and/or changes to the pressure of the drilling fluid.

### EXAMPLE 1

### Disc Shaped Specimen

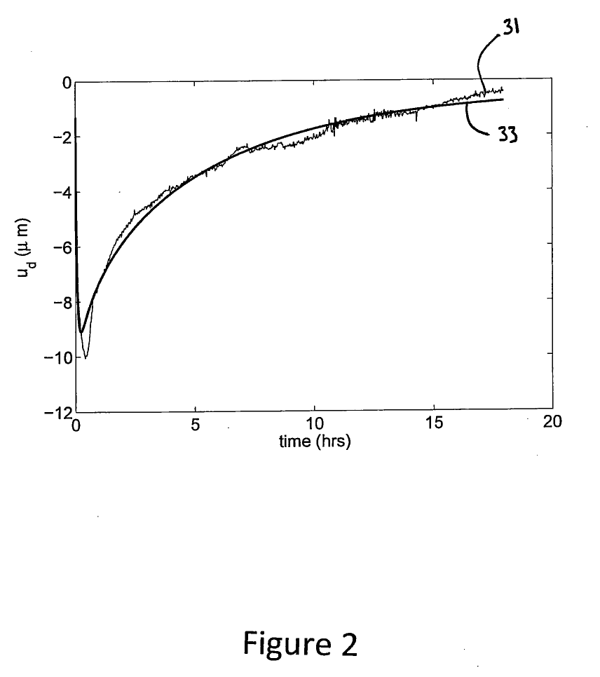

FIG. 2 shows an illustrative example in which the change in the thickness of a 3.19 mm thick shale disc specimen 13 that was subjected to a 1.24 mol/l increase in the salt concentration in the surrounding fluid is recorded (see plotted line 31). This specimen 13 was manufactured from a small chip from the Muderong shale formation by lapping two parallel flat sides using a sanding block.

From FIG. 2 it can be noted that the specimen 13 is shown to shrink by 10 μm over the first hour of the experiment before recovering most, but not all, of the initial deformation over the subsequent 15 or so hours.

The heavy black line 33 gives the results of fitting the solution for a spherical shale ball subjected to the same loading. The mechanical parameters characterising the shale specimen 13 are selected so that the model gives a best fit to the data. The Bayesian (probablistic) method was used, however, a similar result could be obtained with more familiar least-squared fitting.

A few parameters, which are strongly correlated to the others in the model for the shale ball, had to be fixed in order for the fit to be successful. In particular, upon fixing

$$C = 0.004 \frac{1}{MP a} , B = b = 0.986 , β = 0.25 , v = 0.4 ,$$

it was found by a minimization of the mismatch between the model and data that

$${D}_{h} = 4.6 \cdot {10}^{- 9} \frac{{m}^{2}}{s} , {D}_{s} = 0.065 {D}_{h} , \\ R = 0.14 , α = 0.015 , φ = 0.21$$

where the chemical parameter γ is given in terms of the connected porosity φ.

The quality of the fit between the model and data is very good, particularly considering that the solution used in this case is for a spherical ball which is a poor approximation of the experimental geometry.

Ongoing development of more sophisticated solutions will both improve the consistency of results between experiments and will increase the number of parameters which can be independently identified in a given experiment.

From this information recommendations can be made relevant to drilling and wellbore stability. For example, it has been shown that the most important parameter for wellbore stability is the reflection coefficient R.

In this case R=0.14 is a moderate value, indicating that some stabilizing of the wellbore may be possible with the addition of salt to a water-based drilling mud.

Additionally, the chemomechanical coupling parameter α relates to the long term response of the shale. Here α=0.015 is a small value indicating that nearly all of the original shrinkage will eventually be recovered. Based on a characteristic time t_{c}L²/Dₖ, where L is a length of a region of interest, and taking from the experiment Dₕ=4.9·10⁻⁹ m²/s, one may then suggest that the stabilizing effect obtained by adding salt to the drilling fluid can be expected to last for a few months up to a year based on behaviour of the 10-25 cm thick region near the wellbore.

### EXAMPLE 2

### Cylindrical Shaped Specimen

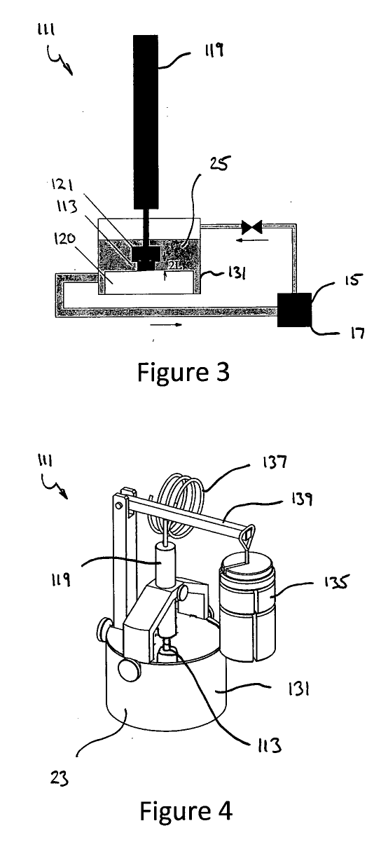

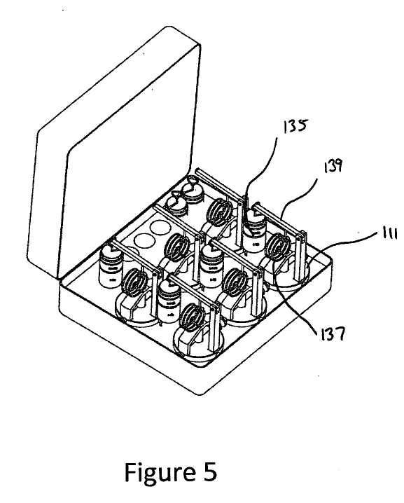

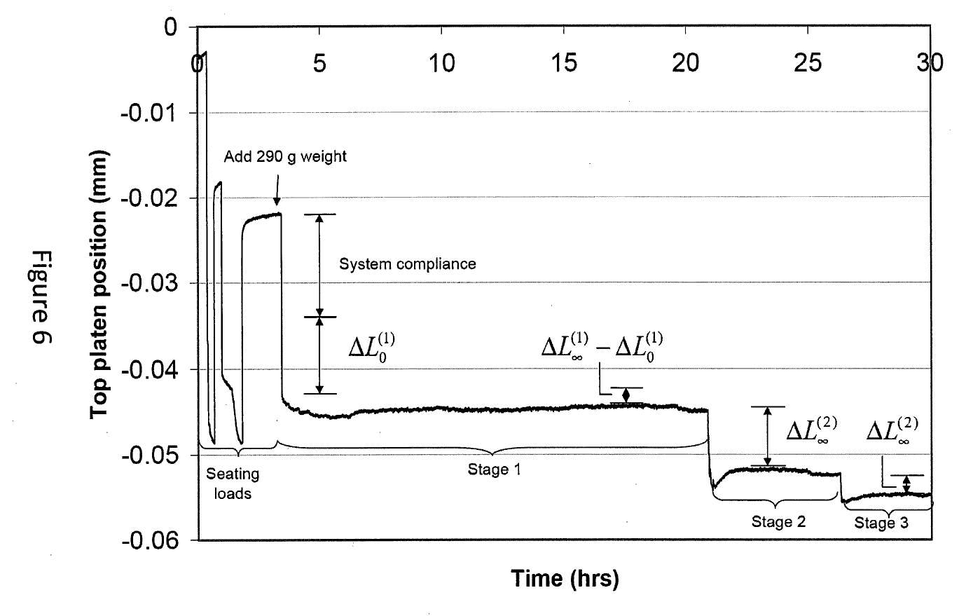

The apparatus 111 used in this second embodiment is similar to that used and described above, therefore same components will be as numbered above. Referring to FIGS. 3 and 4 consider a specimen 113 of Officer Basin shale in the form of a cylinder of length 2L and radius R that is submerged in a vessel 23 containing a well-stirred fluid 25. The fluid is maintained at a constant temperature by a heater 131 contained within the wall of the vessel 23. The specimen 113 is initially equilibrated with the surrounding fluid 25, which in this case is a 3.5% (by weight) solution of NaCl and water that is intended to approximate the salinity of the in situ pore fluid.

The specimen 113 is also initially at equilibrium under a mechanical loading of a 30 gram top platen 121. The displacement of the specimen 113 is measured using a measuring device which in this embodiment is in the form of a linear variable differential transformer 119 (LVDT), as shown in FIG. 3. Furthermore, a small amount of silicone grease is placed between the specimen 113 ends and the lower platen 120 and top platen 121 so that fluid is restricted to flow in the radial direction only.

The displacement of the specimen 113 through successive loading by a weight 135, as recorded by a data acquisition system 137 is shown in FIG. 6. This figure represents movement of the specimen 113 as measured by movement of the top platen 121, versus time. The initial time period of FIG. 6 relates to the specimen 113 equilibrating with the fluid, and the top platen 121 being caused to make good contact with the specimen.

Once good contact has been established between the top platen 121 and the specimen 113, two stages of loading are applied to the specimen 113, which in this example measure 2L=3.88 mm and 2R=3.83 mm. First an additional 290 gram mass is applied to the top platen 121 resulting in a sudden change to axial stress of Δσ_{zz}⁽¹⁾=0.25 MPa (Stage 1). This loading is applied via a weight 135 attached to a load arm 139 which causes the load of the weight 135 to act upon the specimen 113 through the LVDT 119. This uniaxial (Δσᵣᵣ=Δσ_{θθ}=0) mechanical loading phase gives rise to an initial change, known as the undrained response wherein the fluid diffusion has not yet had time to have an effect, in specimen length ΔL₀⁽¹⁾ that is easily obtained from substitution of

$$( \begin{matrix}ɛ \\ ζ \\ θ\end{matrix} ) = ( \begin{matrix}C & bC & - α bC \\ bC & {S}_{σ} & - {S}_{σ} β \\ - α bC & - {S}_{σ} β & γ / N\end{matrix} ) ( \begin{matrix}Δσ \\ Δ p \\ Δπ\end{matrix} ) \qquad ( 10 )$$

with ζ=Δπ=0 and σ=Δσ_{zz}⁽¹⁾/3 into

$$2 G {ɛ}_{ij} = {Δσ}_{ij} - \frac{3 v}{1 + v} {Δσδ}_{ij} + \frac{1 - 2 v}{1 + v} b {δ}_{ij} ( Δ p - α Δπ ) \qquad ( 11 )$$

and integrating according to:

$$Δ L = {\int }_{- L}^{L} {ɛ}_{zz} d z \qquad ( 12 )$$

in order to give:

$$Δ {L}_{0}^{( 1 )} = - \frac{2 LC {Δσ}_{zz}^{( 1 )}}{3} ( \frac{1}{1 - 2 v} - \frac{bB}{3} ) \qquad ( 13 )$$

Additionally, after a period that is typically several hours long, another steady state is attained known as the drained response, wherein the final change in length of the specimen ΔL_{∞}⁽¹⁾ is given by:

$$Δ {L}_{∞}^{( 1 )} = - \frac{2 LC {Δσ}_{zz}^{( 1 )}}{3 ( 1 - 2 v )} \qquad ( 14 )$$

This result is obtained by substituting Equation (10) with Δp=Δπ=0 and Δσ=Δσ_{zz}⁽¹⁾/3 into Equation (11) and integrating the result using Equation (12).

The evolution of the change in length ΔL⁽¹⁾ of the Officer Basin Shale specimen to the dead weight loading is shown in FIG. 6. The loading stage that will be used in analysis is preceded here by two short seating load applications, as previously mentioned. The drained elastic compliance C is first obtained from the change in specimen length between the undrained and drained responses, making use of:

$$Δ {L}_{∞}^{( 1 )} - Δ {L}_{0}^{( 1 )} = - \frac{2}{9} CBbL {Δσ}_{zz}^{( 1 )} \qquad ( 15 )$$

For shales it is typical that B≈b≈1, hence from this experiment it is found that C=0.012 MPa⁻¹. The Poisson's ratio may then be determined from the undrained response ΔL₀⁽¹⁾, making use of Equation (13) to obtain, for the current experiment, ν=0.3. Note that in this case ΔL₀⁽¹⁾ is obtained from the experimental response, taking into account that the compliance of the loading and measuring system leads to a displacement of −0.0134 mm, determined from prior calibration, that is subtracted from the measured response in order to obtain ΔL₀⁽¹⁾. Using C and ν an estimate of the drained elastic Young's modulus is E=100 MPa where it is noted that this value is smaller than would typically be expected and this is due, at least in part, to the fact that the testing is performed using very small loads. Using larger loads or running the tests with an elevated confining pressure would be expected to give larger values of the elastic modulus.

Following the first stage of loading, the concentration of NaCl is suddenly increased by Δcₛ=1.17 mol/litre, which, considering that the experiment was performed at 25° C., corresponds to a change in the osmotic pressure of Δπ⁽²⁾=2.9 MPa (Stage 2). Considering Equation (10) subject to Δσ=Δp=0 and Δπ=Δπ⁽²⁾ leads to the large-time steady-state value of the volumetric strain

ε_{∞}⁽²⁾=−αCbΔπ⁽²⁾ (16)

Classical strain-displacement relations for axisymmetric deformation of a cylinder dictate that

$$ɛ = \frac{\partial {u}_{r}}{\partial r} + \frac{{u}_{r}}{r} + \frac{\partial {u}_{z}}{\partial z} \qquad ( 17 )$$

Further, at large time Δσᵢⱼ=0 so that it is apparent from the extended Hooke's law (Equation 11) that for the second stage of loading

$$\frac{\partial {u}_{r}}{\partial r} = \frac{{u}_{r}}{r} = \frac{\partial {u}_{z}}{\partial z} , t \to ∞ \qquad ( 18 )$$

Hence the large time value of the change in specimen length ΔL_{∞}⁽²⁾ is given directly from integration of Equation (11) according to Equation (12), hence

$$Δ {L}_{∞}^{( 2 )} = - \frac{2 α CbL {Δπ}^{( 2 )}}{3} \qquad ( 19 )$$

From this equation the chemomechanical coupling parameter α can be determined. The large time changes in the specimen length for the osmotic loading are shown in FIG. 6. Note that the shrinking followed by partial recovery is an expected behaviour based on a solution for a spherical shale. The large time response, when substituted into Equation (19), gives the chemomechanical coupling parameter, α=0.12.

As an optional step, a secondary osmotic loading was applied corresponding to Δπ⁽²⁾=1.5 MPa (Stage 3). The response in this case leads to α=0.08. The difference between these two values hints at the expected variability in these experiments. Indeed subsequent experiments indicate that obtaining reliable results will likely require experiments to be performed on approximately 10 specimens, which is enabled by parallel testing systems.

From FIG. 6, two elastic parameters, the drained elastic compliance (C) and Poisson's ratio ν, can be determined from stage 1, whilst the chemomechanical coupling parameter α and reflection coefficient R can then be determined from stage 2. Stage 3 verifies the findings of stage 2.

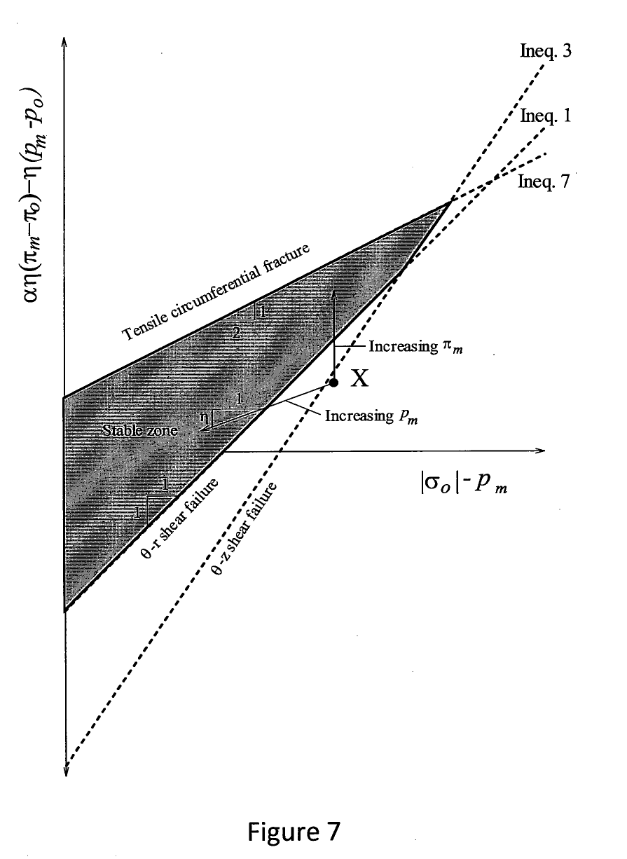

Once the specimen 113 is characterised and the chemomechanical coupling parameter α and reflection coefficient R are determined, this information can be used to predict how changes to the salt concentration in the drilling fluid can, for instance, influence the stress concentration at the borehole wall. Consider FIG. 7 which represents a wellbore stability model. In this model the abscissa represents the difference between the isotropic insitu stress σₒ (tension is positive) and drilling fluid pressure pₘ, whilst the ordinate represents the combination of the hydraulic and osmotic pressure initially characterizing the rock and for the drilling fluid in the form that it enters the stability criterion.

Knowing the current drilling conditions and the chemoporomechanical parameters of the rock from specimen 113, the location of the initial evaluation point ‘X’ can be plotted on the model. From this the drilling engineer can determine the required increase in salt πₘ in the drilling fluid and/or increase in drilling fluid pressure pₘ in order to move the evaluation point ‘X’ to a position in which the conditions at the borehole wall will be stable for that particular formation of shale.

The hydraulic and osmotic drilling fluid pressures are the quantities that are the most readily accessible to the drilling engineer as these quantities can be altered by changing the density and salt content of the drilling fluid, respectively. Changing these quantities will cause a shift in the location of the initial evaluation point ‘X’. For example, increasing the drilling fluid density, and hence pₘ, will cause a decrease in both the ordinate and abscissa values along a trajectory with a slope equal to η (which is typically about 0.2 for shales). On the other hand, increasing the salt content of the drilling fluid, and hence πₘ, will lead to a vertical shift in the evaluation point.

It is particularly useful to note that, while in some cases wellbore stability will be improved by adding salt to the drilling fluid (i.e. the point will be moved inside the stable zone from a zone characterised by shear failure around the wellbore), in other cases, salting the drilling fluid could lead to the formation of tensile circumferential fractures. Discernment between these cases depends critically on the chemoporoelastic characterisation of the formation. Hence, it is clear that rapid characterisation of the relevant shale parameters has the potential to drastically improve the reliability with which wellbore stability related problems are addressed.

Once the rock has been characterised and the chemoporoelastic parameters of the rock are known, the parameters can also be placed in a model which relates to the conditions of the wellbore wall away from its surface. This will help in determining the long term stability of the wellbore and how this will be affected by changes in the salt content and pressure of the drilling fluid.

This invention provides a method for determining the relevant chemoporoelastic parameters based on measuring the geometric (e.g. length) changes of a specimen that is subjected to both mechanical (weight) and chemical (ionic) loading. The results, combined with a wellbore stability analysis, provide a new drilling rig-based procedure that will allow drilling engineers to improve wellbore stability by making changes to drilling fluid density and salt content that take into account the unique properties of each problematic shale formation that is encountered during drilling operations.

Modifications and variations such as would be apparent to the skilled addressee are considered to fall within the scope of the present invention.

Throughout the specification, unless the context requires otherwise, the word “comprise” or variations such as “comprises” or “comprising”, will be understood to imply the inclusion of a stated integer or group of integers but not the exclusion of any other integer or group of integers.

1. A method for measuring the change in behaviour of a rock specimen in a fluid of prescribed composition, wherein the rock specimen is prepared from drill cuttings, wellbore cave-in material, or core offcuts the method comprises:
stirring the fluid so that the salinity level of fluid remains substantially uniform;
controlling the temperature at which the experiment is conducted such that it is maintained within the desired range;
applying at least one load to the specimen;
measuring the deformation of the specimen in response to the change in the at least one load.

2. The method according to claim 1 comprises an initial step of preparing a specimen from drill cuttings or other small shale fragments.

3. The method according to claim 2 wherein the specimen is a regular geometrical shape.

4. The method according to claim 1 wherein the fluid initially approximates the composition of the specimens natural pore fluid

5. The method according to claim 1 comprising a further initial step of allowing sufficient time for the specimen to equilibrate with the fluid.

6. The method according to claim 5 wherein the specimen is left for sufficient time for the specimen to reach equilibrium, evidenced by the length of the specimen remaining substantially constant.

7. The method according to claim 1 wherein the load upon the specimen is changed by applying a chemical and/or mechanical load thereto.

8. The method according to claim 7 wherein chemical loading is applied by changing the chemical composition of the fluid.

9. The method according to claim 7 wherein comprising the step of adding salt over a period of time to alter the chemical loading by changing the ionic concentration.

10. The method according to claim 9 wherein salt is added over a period of time in the order of 30-60 seconds.

11. The method according to claim 10 wherein further salt is added and measurements continue to be taken over the next 5 to 15 hours.

12. The method according to claim 1 where a solution for the measured deformation of the specimen in response to the change in that at least one load is developed for the specimen using a chemoporoelastic model.

13. The method according to claim 7 wherein a force applied to the specimen creates the mechanical loading, whereby the force is applied by a weight applied to the specimen.

14. The method according to claim 13 wherein the mechanical loading begins with a series of axial loading such that contact between the specimen and loading surface is sufficiently established.

15. The method according to claim 13 wherein the mechanical loading comprises applying a load, measuring the initial elastic response and measuring the change in displacement over the next one to three hours.

16. The method according to claim 1 wherein the temperature is maintained a few degrees above ambient or at the in situ temperature of the shale formation.

17. The method according to claim 1 wherein the load upon the specimen is changed by applying a hydraulic load to the specimen.

18. A method of predicting the behaviour of a rock formation when exposed to changes in chemical composition and/or pressure of a drilling fluid, the method comprising:
using a portable apparatus measure the displacement of a specimen of the rock formation in a fluid over a period of time in which a load is applied thereto, that load being in the form of a mechanical force applied to the specimen and/or the addition of salt to the fluid;
using these measurements and known formula to determine various chemoporomechanical properties of the rock formation;
using formulas that predict wellbore stability and depend on the determined chemoporomechanical properties of the rock formation in order to recommend optimal modifications to the salt content and/or density of the drilling fluid in order to promote wellbore stability.

19. A method to predict the stability of the rock formation surrounding a wellbore, the method comprises the steps of:
expressing the experimental data in terms of appropriate quantities in an experimental configuration;
solving a chemoporoelastic boundary value problem relevant to the experimental configuration;
employing said solution in an inverse problem to select material parameters which give the best agreement between the experimental configuration results and the experimental data;
evaluating the uncertainty associated with said parameter estimates and possible covariance between experimental configuration parameters for a given experiment so that the possible error introduced into the wellbore stability calculation by this uncertainty can be quantified; and
using a wellbore stability analysis to provide recommendations regarding the anticipated response of the rock formation to the addition of salt to the drilling fluid.

20. A portable apparatus to measure the change in behaviour of a rock specimen in a fluid of prescribed composition, wherein the rock specimen is prepared from drill cuttings, wellbore cave-in material, or core offcuts, the apparatus comprises:
a vessel in which the fluid and specimen are placed;
a loading means for applying at least one load upon the specimen;
at least one measuring device to measure the deformation of the specimen in response to a change in load;
a stirring system to maintain uniformity of the fluid;
a temperature regulating device to maintain the fluid at the desired temperature.

21. The apparatus according to claim 20 wherein the apparatus further comprises a data acquisition system to record the data relating to the deformation of the specimen.

22. The apparatus according to claim 20 wherein the apparatus is self contained and autonomous.

23. The apparatus according to claim 20 wherein the measuring device and data acquisition system is shielded to prevent noise from other devices affecting the data.

24. The apparatus according to claim 20 wherein the specimen has one or more axis of symmetry.

25. The apparatus according to claim 20 wherein the specimen is a regular geometrical shape, such as a cylinder.

26. The apparatus according to claim 25 wherein the specimen is cylindrical, having a diameter of approximately 4 mm.

27. The apparatus according to claim 20 wherein the specimen is first prepared in a jig from a drill cutting.

28. The apparatus according to claim 20 wherein the loading means is in the form of a chemical and/or mechanical load to the specimen.

29. The apparatus according to claim 28 wherein the chemical load is applied by changing the ionic concentration of the fluid, such as by adding salt to the fluid.

30. The apparatus according to claim 28 wherein the mechanical load is applied by applying a force to the specimen.

31. The apparatus according to claim 30 wherein the mechanical load is applied by applying a weight axially with respect to the specimen.

32. The apparatus according to claim 20 wherein the loading means is in the form of a hydraulic load applied to the specimen.

33. The apparatus according to claim 20 wherein the at least one measuring device is capable of measuring a micrometer-scale response on a specimen less than 5 mm in thickness.

34. The apparatus according to claim 33 wherein the at least one measuring device comprises a linear variable differential transformer.

35. The apparatus according to claim 34 wherein the linear variable differential transformer comprises a loading platen through which the mechanical loading may be applied to the specimen.

36. The apparatus according to claim 35 wherein the loading platen is connected to a loading arm to which the desired weight may be applied.

37. The apparatus according to claim 20 wherein the temperature regulating device comprises heat elements located in the wall of the vessel.

38. The apparatus according to claim 20 wherein the temperature regulating device comprises heat elements located in the fluid.

39. A portable measurement apparatus which can be used in the laboratory or transported to drilling sites in order to perform on-site characterisation of shale formations wherein the apparatus includes a means for applying a prescribed ionic, mechanical and/or hydraulic loading to specimens with stirring and temperature regulation.

40. The portable measurement apparatus according to claim 39 wherein the measurements recorded by the apparatus are interpreted in order to provide detailed characterisation of the chemoporoelastic parameters associated with the shale formation.

41.-42. (canceled)
